(19) United States
(12) Patent Application Publication
(10) Pub. No.: US 2022/0175885 A1
(43) Pub. Date: Jun. 9, 2022

(54) TARGETING BIOLOGICAL AGENTS TO MUCOSAL DEFECTS OF THE GASTROINTESTINAL TRACT

(71) Applicant: Reponex Pharmaceuticals A/S, Hørshoim (DK)

(72) Inventors: Lars Otto Uttenthal, Madrid (ES); Lasse Lindblad, Ålsgårde (DK)

(21) Appl. No.: 17/439,339

(22) PCT Filed: Mar. 19, 2020
(86) PCT No.: PCT/EP2020/057575

§ 371 Date: Sep. 14, 2021

(30) Foreign Application Priority Data
Mar. 20, 2019 (DK) PA201970173
May 22, 2019 (DK) PA201970324

(51) Int. Cl. A61K 38/19; A61K 38/28; A61K 38/22; A61K 38/17; C07K 16/24; A61K 9/00; A61K 31/7032; A61K 33/08

(52) U.S. Cl. CPC A61K 38/193; A61K 38/28; A61K 38/2264; A61K 38/1793; C07K 2317/24; A61K 9/0053; A61K 31/7032; A61K 33/08; C07K 16/241

(57) ABSTRACT

Compositions are provided that target biological therapeutic agents to mucosal defects in the gastrointestinal tract by means of antacid mucosal protective agents that bind selectively to such defects.

### FIELD OF INVENTION

The present invention provides compositions that target therapeutic biological agents to lesions involving mucosal defects of the gastrointestinal tract, which may be peptic ulcers or ulcerative or fistular lesions arising from inflammatory bowel diseases, in particular Crohn's disease, thus providing a more effective means of delivering these agents locally to the lesions from the bowel lumen and ensuring longer contact of the agents with lesions, while reducing the contact of the agents with healthy bowel mucosa. As such, it is particularly relevant to the field of gastroenterology.

### BACKGROUND OF THE INVENTION

There are a number of substances that are known to bind with some degree of selectivity to mucosal defects of the gastrointestinal tract. These defects may be gastric or duodenal ulcers, which are those that have been most studied, but may also be defects due to mucosal resection, lesions due to inflammatory bowel diseases such as Crohn's disease, or ulcers arising from radiation or cytotoxic injury to the bowel mucosa. The mechanism of selective binding is presumed to involve the greater binding of these substances to proteins on the surface of the tissue exposed by the mucosal defect, which may be termed, when appropriate, “the ulcer base”, than to the mucus covering the surrounding healthy mucosa. These substances are also capable of non-covalently binding individual proteins or peptides, in vitro as in vivo, and in some cases it has been found that the bound proteins are protected from denaturation or degradation by exposure to acid, such as gastric acid, or to denaturing agents, such as urea, or thermic denaturation, or the action of proteolytic enzymes, and retain their biological activity. Many of the binding substances in question are broadly termed “antacids” and/or “mucosal protective agents” and have been used to treat peptic ulcers. Principal among them is sucralfate, the aluminum salt of sucrose octasulfate, which is almost completely insoluble in aqueous media at neutral or physiological pH, but may be solubilized by dilute acids, such as gastric acid, in which it forms a gel, or by dilute alkalis. Sucralfate binds selectively to ulcers of the stomach and duodenum, so that its binding to the ulcer base is between 6- to 7-fold (e.g. Nakazawa et al 1981) and 12- to 18-fold (Itoh et al 2004) greater than its binding to the surrounding healthy mucosa, while this selective binding may be maintained for 6 hours (Giesing et al 1981) to 12 hours (Itoh et al 2004). Further, the selective binding does not appear to depend on a low pH, but occurs also at pH values at around 8 (Itoh et al 2004). It has also been shown to bind a variety of proteins by non-covalent means.

### SUMMARY OF THE INVENTION

While the properties of sucralfate have been studied with respect to its action in situ to potentiate the effects of endogenous signaling proteins secreted from lesions involving mucosal defects of the gastrointestinal mucosa, the present invention exploits these properties by using sucralfate as a carrier substance to preserve and target exogenously applied therapeutic proteins or peptides to these lesions, so that these exogenous substances can act on the lesions at an effective concentration even when they are applied via the lumen of the gastrointestinal tract at a level that is not directly over the lesions. While sucralfate is the paradigm for such carrier substances of the present invention, other substances, particularly from the class of agents termed “antacids” and/or “mucosal protective agents”, may have broadly similar properties in being able to bind both to proteins exposed by mucosal defects and to exogenous therapeutic proteins or peptides. Common to these agents is that they all contain or are chemically derived from aluminum hydroxide. Such carrier substances include aluminum hydroxide gels (e.g. algeldrate, Alhydrogel), which may be chemically combined with magnesium hydroxide, as in magaldrate gels and hydrotalcite suspensions, or mixed with magnesium hydroxide, as in Maalox.

Accordingly, the present invention provides a composition comprising an exogenous protein therapeutic agent selected from granulocyte-macrophage colony-stimulating factor, insulin, leptin, infliximab, adalimumab, certolizumab, golimumab or etanercept for use in administering via the gastrointestinal lumen said exogenous protein therapeutic agent to a mucosal defect located in the esophagus, stomach or intestines, said mucosal defect comprising an esophageal ulcer, a peptic ulcer of the stomach or duodenum, an ulcer due to Helicobacter pylori infection, a mucosal defect due to surgical or endoscopic mucosal resection, an inflammatory ulcer due to Crohn's disease or ulcerative colitis, a fistula due to Crohn's disease, or an ulcer due to radiation damage or the use of cytotoxic drugs, wherein the exogeneous protein therapeutic agent is bound to an antacid mucosal protective agent as a carrier substance which selectively binds to said mucosal defect and which is selected from sucralfate, aluminum hydroxide gel, magaldrate gel, or hydrotalcite, which are in aqueous suspension.

In one embodiment, the aqueous suspension additionally contains one or more pharmaceutically acceptable thickening agents for adjusting viscosity.

In further embodiments, the composition has been formulated for administration by oral ingestion, by enema, or by endoscopic delivery into the lumen of the gastrointestinal tract.

In another embodiment, the present invention provides a kit of parts for preparing a composition for use according to the present invention, which kit of parts consists of a) an aqueous suspension of the carrier substance; b) a stable preparation of the protein therapeutic agent; and c) a diluent for the protein therapeutic agent and for diluting the composition after the carrier substance and protein therapeutic agent have been mixed.

In a further embodiment, the present invention provides a method for delivering a therapeutic composition comprising an exogenous protein therapeutic agent via the gastrointestinal lumen to a mucosal defect of the esophagus, stomach or intestines, said method comprising oral, rectal or endoscopic administration of the composition comprising the exogenous protein therapeutic agent bound to an antacid mucosal protective agent as a carrier substance, which is in aqueous suspension.

In one embodiment, the antacid mucosal protective agent, which is in aqueous suspension, is selected from sucralfate, an aluminum hydroxide gel, a magaldrate gel, and/or a hydrotalcite.

In another embodiment, the exogenous protein therapeutic agent bound to the carrier substance is granulocyte-macrophage colony-stimulating factor.

In another embodiment, the exogenous protein therapeutic agent bound to the carrier substance is granulocyte-macrophage colony-stimulating factor and the carrier substance is sucralfate in aqueous suspension.

In another embodiment, the exogenous protein therapeutic agent bound to the carrier substance is selected from insulin, leptin, infliximab, adalimumab, certolizumab, golimumab, or etanercept.

In another embodiment, the aqueous suspension further contains one or more pharmaceutically acceptable thickening agents for adjusting viscosity.

In another embodiment, the therapeutic composition has been formulated for administration by oral ingestion, by enema, or by endoscopic delivery into the lumen of the gastrointestinal tract.

In another embodiment, the mucosal defect to be treated is selected from an esophageal ulcer, a peptic ulcer of the stomach or duodenum, or an ulcer due to Helicobacter pylori infection, a defect due to surgical or endoscopic mucosal resection, an inflammatory ulcer due to Crohn's disease or ulcerative colitis, a fistula due to Crohn's disease, or an ulcer due to radiation damage or the use of cytotoxic drugs.

Accordingly, the present invention provides a composition comprising an antacid mucosal protective agent as a carrier substance to which an exogenous biological therapeutic agent, typically a protein or peptide, has been bound, for use in targeting said biological agent to areas of mucosal defect in the gastrointestinal tract when administered via the gastrointestinal lumen.

In principle, the biological therapeutic agent bound to the carrier substance can be any agent that binds to the carrier and retains its biological activity once the carrier-agent complex binds to the tissue exposed by the mucosal defect.

In another embodiment, the composition for use according to the present invention is formulated as an aqueous suspension, with or without the addition of pharmaceutically acceptable thickening agents.

In another embodiment, a composition is disclosed that comprises an exogenous protein or peptide biological therapeutic agent for use according to some of the previous embodiments, wherein the biological therapeutic agent bound to the carrier substance is selected from agents comprising the cytokines interleukin 1α, interleukin 1β, interleukin 6, interleukin 10 and tumor necrosis factor α, the growth factors of the platelet-derived, fibroblast, epidermal, vascular endothelial, insulin-like, plasminogen-related and connective tissue families, nerve growth factor, members of the transforming growth factor β superfamily, and the hormones insulin and leptin, or any combination thereof.

In another embodiment, a composition is disclosed for use according to any of the preceding embodiments, wherein the biological therapeutic agent is GM-CSF and the carrier substance is a sucralfate suspension.

In another embodiment, the method according to some of the previous embodiments is disclosed, wherein the biological therapeutic agent bound to the carrier substance is selected from the cytokines interleukin 1α, interleukin 1β, interleukin 6, interleukin 10 and tumor necrosis factor α, the growth factors of the platelet-derived, fibroblast, epidermal, vascular endothelial, insulin-like, plasminogen-related and connective tissue families, nerve growth factor, members of the transforming growth factor β superfamily and the hormones insulin and leptin, or any combination thereof.

In another embodiment, the method according to the previous embodiments is disclosed, wherein the mucosal defect to be treated is selected from an esophageal ulcer, a peptic ulcer of the stomach or duodenum, or an ulcer due to Helicobacter pylori infection, a defect due to surgical or endoscopic mucosal resection, an inflammatory ulcer due to Crohn's disease or ulcerative colitis, a fistula due to Crohn's disease, or an ulcer due to radiation damage or the use of cytotoxic drugs.

In the following detailed description of the invention, details of the scope of the invention will be given, together with details of the practical performance of the invention.

### DETAILED DESCRIPTION OF THE INVENTION

In describing the embodiments of the invention, specific terminology will be resorted to for the sake of clarity. However, the invention is not intended to be limited to the specific terms so selected, and it is understood that each specific term includes all technical equivalents which operate in a similar manner to accomplish a similar purpose.

When describing the embodiments of the present invention, the combinations and permutations of all possible embodiments have not been explicitly described. Nevertheless, the mere fact that certain measures are recited in mutually different dependent claims or described in different embodiments does not indicate that a combination of these measures cannot be used to advantage. The present invention envisages all possible combinations and permutations of the described embodiments.

The terms “comprising”, “comprise” and “comprises” herein are intended by the inventors to be optionally substitutable with the terms “consisting of”, “consist of” and “consists of”, respectively, in every instance.

As used herein, the singular forms “a”, “an” and “the” are intended to include the plural forms as well, unless the context clearly indicates otherwise.

Carrier Substances Targeting Mucosal Defects

Sucralfate: To be pharmaceutically acceptable, this is selected to meet the specifications of the European and United States pharmacopoeias with the additional requirement that the particle size is such that the equivalent spherical diameter is within the range of 2-100 μm, preferably with the median equivalent spherical diameter within the range of 3-10 μm. This is to provide a high surface area for the efficient adsorption of the biological agents to be bound to it. The range of concentrations of sucralfate in the formulated composition is from 5% to 40% (w/v), preferably 10% to 20% (w/v).

Aluminum hydroxide gel: This is also selected to meet the specifications of the European and United States pharmacopoeias and the preferred range of particle sizes is the same as that specified for sucralfate. The range of concentrations of aluminum hydroxide in the formulated composition is from 1% to 4% (w/v) assayed as Al(OH)₃, or with an aluminum content of 0.5% to 2% (w/v). A preferred concentration is 2% (w/v) Al(OH)₃ containing approximately 1% (w/v) aluminum.

Magaldrate gel: This is also selected to meet the specifications of the European and United States pharmacopoeias and the preferred range of particle sizes is the same as that specified for sucralfate. The range of concentrations of magaldrate in the formulated composition is from 5% to 20% (w/v), preferably 10% (w/v).

Hydrotalcite suspension: The preferred range of particle sizes is the same as that specified for sucralfate. The range of concentrations of hydrotalcite in the formulated composition is from 5% to 20% (w/v), preferably 10% (w/v).

Biological Therapeutic Agents to be Bound to the Carrier Substances

In principle, the biological therapeutic agent bound to the carrier substance can be any agent that binds to the carrier and retains its biological activity once the carrier-agent complex binds to the tissue exposed by the mucosal defect. These will chiefly be proteins or peptides and the purpose of targeting them to the mucosal defects will be to promote the healing of these defects when applied exogenously via the bowel lumen, while reducing wastage of the agents by degradation during their passage down the gastrointestinal tract. Exogenously applied proteic biological agents that may promote the healing of mucosal defects, whether these are caused by the actions of gastric acid, bile salts, Helicobacter pylori infection, the inflammatory processes of Crohn's disease or ulcerative colitis, or the actions of radiation and/or cytotoxic drugs, include the following cytokines, growth factors and hormones, which may act at various stages of the healing process: GM-CSF, IL-1α, IL-1β, IL-6, IL-10, TNF-α, the PDGF, FGF, EGF, VEGF, IGF, PRGF and CTGF/CNN families, as well as NGF and members of the TGF-β superfamily, and insulin and leptin. These will typically be produced by recombinant DNA technology and presented in pure form for exogenous application to the lesions. Other biological agents that can be used include therapeutic antibodies or other non-native proteins, such as infliximab, adalimumab, certolizumab, and golimumab, or the fusion protein etanercept.

Binding the Biological Agents to the Carrier Substances

The binding of the biological agents to the carrier substances is a near-instantaneous process at room temperature or 2-8° C., only slowed by the viscosity of the medium and the diffusion distance from molecules of agent to particles of carrier. Essentially the carrier is made up in an aqueous medium, which may be pure water or dilute saline solutions up to a concentration of 155 mM sodium chloride. The pH is adjusted to a value between 7 and 4 with small volumes of 100 mM hydrochloric acid or 100 mM sodium hydroxide. The biological agent is made up in a similar aqueous medium. Any phosphate buffer should as far as possible be avoided, as the carriers absorb phosphate with a corresponding fall in their protein binding capacity. The carrier suspension and agent solutions are mixed in a molar proportion of agent to carrier that does not exceed 1 to 400 or about 1 mg per kDa of molecular weight of the agent per gram of carrier and is no less than 1 to 400,000 or about 1 μg per kDa of molecular weight of the agent per gram of carrier. The actual proportion of agent to carrier that is used will be determined by the amount of agent that it is desired to administer in a given volume of formulated composition containing a given concentration of carrier. The agent and carrier are typically mixed at room temperature or 2-8° C. and the containing vessel is rotated end-over-end for 30 minutes. However, binding often reaches equilibrium within 5 minutes, after which very little agent can be measured in the supernatant. It is usually unnecessary to separate the small proportion of unbound agent. This will remain in the liquid phase of subsequent formulations and will go to waste when the formulation is administered into the bowel lumen.

Formulations

The complexes of the biological agents with the carrier substances of the present invention may be stable for at least 24 hours at pH 6, but their longer-term stability has not been determined. The formulation of the compositions of the invention is therefore recommended to be performed immediately (or within 1 or 2 hours) before their administration into the lumen of the gastrointestinal tract. This process may be facilitated by providing the individual components of the formulation as a kit of parts: one container contains a suspension of the carrier substance in pH-adjusted dilute saline at a concentration that is higher than its desired concentration in the final formulation; a second container contains the freeze-dried biological therapeutic agent; a third container contains a dilute saline solution which is used a) to reconstitute the freeze-dried biological agent so that the calculated amount of it can be added to the suspension of carrier substance by means of a syringe or pipette, and b) to dilute the complex, once formed, to a volume that is suitable for the proposed mode of administration and contains a suitable concentration of carrier for such administration. In some cases, the third container can be replaced by two containers, one containing saline for reconstituting the biological agent, and the second containing an appropriate diluent for the complex, which may optionally include any of a number of pharmaceutically acceptable thickening agents known to the art that are compatible with the complex, as well as pharmaceutically approved flavoring and/or coloring agents to render the final formulation more suitable for oral administration.

Solutes that may be added to water or saline in the above solutions include pH-adjusting agents such as hydrochloric acid, sodium hydroxide and biocompatible buffering agents, excluding phosphate buffers.

The preparation and formulation of a single dose of GM-CSF bound to sucralfate for endoscopic administration to a Crohn's lesion of the cecum or ascending colon is described in Example 1 below.

Administration

The compositions of the present invention are administered via the lumen of the gastrointestinal tract, at the level of, or proximal to, the lesions to be treated. The advantage of using the compositions is precisely that of targeting the biologically active agents to the lesions, so that they do not need to be administered directly over or onto the lesions. Administration may therefore be by oral ingestion, or by endoscopic delivery into the stomach or small intestine or colon, or by enema into the rectum or colon. In each case, the details of the formulation are adjusted so that the volume to be delivered or ingested contains the effective amount of agent/carrier complex with the appropriate viscosity for the method of delivery. The effective amount of carrier may be from 1 to 10 g per single dose.

Indications

The indication for use of the compositions of the present invention is the presence of one or more defects of the lining of the gastrointestinal tract that are considered to require treatment with a biological therapeutic agent that binds to the carrier substance. Such defects may be esophageal ulcers, peptic ulcers of the stomach or duodenum, or ulcers due to Helicobacter pylori infection, defects due to surgical or endoscopic mucosal resection, inflammatory intestinal ulcers due to inflammatory bowel diseases such as Crohn's disease or ulcerative colitis, or fistulae associated with Crohn's disease. They may also be ulcers caused by damage to the mucosa caused by radiation or the use of cytotoxic drugs.

Dose and Dosage Regimens

By “effective amount” of the compositions of the present invention is meant a dose, which, when administered to a subject in need thereof, achieves a concentration of biological therapeutic agent at the mucosal defects to be treated that contributes to an acceleration its healing. Such an effective amount may be determined by physicians of ordinary skill in the art attending patients with such mucosal defects, and will require an accumulation of experience through clinical testing. An effective single dose of GM-CSF in the form of molgramostim may be in the range of 250 μg to 1 mg. It also may be 300 μg, 500 μg, 750 μg, or 900 μg. An effective single dose of GM-CSF in the form of molgramostim may be bound to, for example, 5 g of sucralfate. In a preferred embodiment, an effective single dose of GM-CSF bound to sucralfate is in the range of 250 μg to 1 mg of molgramostim bound to 5 g of sucralfate.

The effective amounts and dosages of the ingredients of the composition are not determined in relation to body weight or body surface area, because the treatment is local to the areas of the gastrointestinal tract that are affected by any mucosal defect. Systemic absorption of either components derived from the carrier substances or the biological therapeutic agents will be minimal, thus avoiding the risk of serious systemic side effects.

Because the compositions of the present invention ensure prolonged contact of the biological agent with the mucosal defect, and the biological action of the agent is typically prolonged for many hours or a period of days after direct contact has ceased, it will not be necessary to administer single doses at a higher frequency than once daily. However, oral doses can be repeated twice or more times a day, if clinician so determines. Oral treatment can be continuous for 1 or 2 weeks, followed by assessment of effect and resumption or discontinuance of treatment as the clinician determines. Administration by enema can also be on a daily basis, but a higher frequency will be less acceptable to the patient and attending staff. Endoscopic administration can in practice only be performed intermittently, but such administration can be supplemented with oral or enema treatment between follow-up endoscopies.

### Example 1

The following example illustrates the preparation of a composition of the invention which contains 5 g of sucralfate and 250 μg of molgramostim (recombinant human GM-CSF expressed in E. coli) in a total volume of 50 mL for administration into the bowel lumen as a single dose via an endoscope. The procedure may be carried out at either room temperature (25° C.) or at 2-8° C.

1. 5 g of sucralfate Ph.Eur. of particle size 5-10 μm are suspended in 25 mL of 0.9% (w/v) saline and the pH adjusted, if necessary, to between 4 and 7.

2. 250 μg of molgramostim from a stock solution containing approximately 2 mg/mL in 5 mM sodium phosphate buffer, is diluted into 5 mL of 0.9% (w/v) saline.

3. The sucralfate suspension and molgramostim solution are mixed in a 100-mL capped polypropylene tube and rotated end-over-end for 5 minutes.

4. The mixture is then diluted to 50 mL with 0.9% (w/v) saline and is ready for administration.

### REFERENCES

Giesing D H, Bighley L D, Iles R L (1981) Effect of food and antacid on binding of sucralfate to normal and ulcerated gastric and duodenal mucosa in rats. J Clin Gastroenterol 3(Suppl 2):111-116.
Itoh T, Kusaka K, Kawaura K, Kashimura K, Yamakawa J, Takahashi T, Kanda T (2004) Selective binding of sucralfate to endoscopic mucosal resection-induced gastric ulcer: evaluation of aluminium adherence. J Int Med Res 32:520-529.
Nakazawa S, Nagashima R, Samloff I M (1981) Selective binding of sucralfate to gastric ulcer in man. Dig Dis Sci 26:297-300.

1. A method of treating a mucosal defect located in the esophagus, stomach or intestines of a subject comprising:
administering via the gastrointestinal lumen to a subject in need thereof a composition comprising an exogenous protein therapeutic agent selected from granulocyte-macrophage colony-stimulating factor, insulin, leptin, infliximab, adalimumab, certolizumab, golimumab or etanercept to a mucosal defect located in the esophagus, stomach or intestines of said subject, said mucosal defect comprising an esophageal ulcer, a peptic ulcer of the stomach or duodenum, an ulcer due to Helicobacter pylori infection, a mucosal defect due to surgical or endoscopic mucosal resection, an inflammatory ulcer due to Crohn's disease or ulcerative colitis, a fistula due to Crohn's disease, or an ulcer due to radiation damage or the use of cytotoxic drugs, wherein the exogeneous protein therapeutic agent is bound to an antacid mucosal protective agent, which selectively binds to said mucosal defect and, which is selected from sucralfate, aluminum hydroxide gel, magaldrate gel, or hydrotalcite, and which are in an aqueous suspension.

2-12. (canceled)

13. The method according to claim 1, wherein the aqueous suspension further comprises one or more pharmaceutically acceptable thickening agents.

14. The method according to claim 1, wherein said composition has been formulated for administration by oral ingestion, by enema, or by endoscopic delivery into the lumen of the gastrointestinal tract.

15. A kit comprising:
a) an aqueous suspension comprising sucralfate, aluminum hydroxide gel, magaldrate gel, or hydrotalcite;
b) a stable preparation comprising granulocyte-macrophage colony-stimulating factor, insulin, leptin, infliximab, adalimumab, certolizumab, golimumab or etanercept; and
c) a diluent suitable for dilution of the aqueous suspension and the stable preparation after the aqueous suspension and stable preparation have been mixed.

16. A method for delivering a therapeutic composition comprising an exogenous protein therapeutic agent via the gastrointestinal lumen to a mucosal defect of the esophagus, stomach or intestines of a subject, said method comprising oral, rectal or endoscopic administration of a composition comprising an exogenous protein therapeutic agent bound to an antacid mucosal protective agent, which is in aqueous suspension, via the gastrointestinal lumen, to a mucosal defect of the esophagus, stomach or intestines of said subject.

17. The method according to claim 16, wherein the antacid mucosal protective agent, which is in aqueous suspension, is selected from sucralfate, an aluminum hydroxide gel, a magaldrate gel, and/or a hydrotalcite.

18. The method according to claim 16, wherein the exogenous protein therapeutic agent bound to the carrier substance is granulocyte-macrophage colony-stimulating factor.

19. The method according to claim 16, wherein the exogenous protein therapeutic agent bound to the antacid mucosal protective agent is granulocyte-macrophage colony-stimulating factor and the antacid mucosal protective agent is sucralfate in aqueous suspension.

20. The method according to claim 16, wherein the exogenous protein therapeutic agent bound to the antacid mucosal protective agent is selected from insulin, leptin, infliximab, adalimumab, certolizumab, golimumab, or etanercept.

21. The method according to claim 16, wherein the composition further comprises one or more pharmaceutically acceptable thickening agents.

22. The method according to claim 16, wherein the therapeutic composition has been formulated for administration by oral ingestion, by enema, or by endoscopic delivery into the lumen of the gastrointestinal tract.

23. The method according to claim 16, wherein the mucosal defect is selected from an esophageal ulcer, a peptic ulcer of the stomach or duodenum, or an ulcer due to Helicobacter pylori infection, a defect due to surgical or endoscopic mucosal resection, an inflammatory ulcer due to Crohn's disease or ulcerative colitis, a fistula due to Crohn's disease, or an ulcer due to radiation damage or the use of cytotoxic drugs.
